(19) United States
(12) Patent Application Publication
(10) Pub. No.: US 2024/0324906 A1
(43) Pub. Date: Oct. 3, 2024

(54) DETERMINATION METHOD, DETERMINATION DEVICE, AND DETERMINATION SYSTEM

(71) Applicant: Panasonic Intellectual Property Management Co., Ltd., Osaka (JP)

(72) Inventors: Kengo WADA, Osaka (JP); Yoshihiro MATSUMURA, Osaka (JP); Takahiro AIHARA, Osaka (JP); Taichi HAMATSUKA, Osaka (JP); Fumihiro NARUSE, Kyoto (JP); Chie MINAMI, Kyoto (JP); Ryota SUZUKI, Kyoto (JP)

(73) Assignee: Panasonic Intellectual Property Management Co., Ltd., Osaka (JP)

(21) Appl. No.: 18/290,767

(22) PCT Filed: May 25, 2022
(86) PCT No.: PCT/JP2022/021370

§ 371 Date: Jan. 20, 2024

(30) Foreign Application Priority Data
Jul. 28, 2021 (JP) 2021-122906

(51) Int. Cl. A61B 5/11; G06V 20/64; G06V 40/20; G16H 20/30

(52) U.S. Cl. CPC A61B 5/1118; A61B 5/1128; G06V 20/64; G06V 40/23; G16H 20/30

(57) ABSTRACT

A determination method is a determination method performed by a computer, and includes: instructing a target person to perform a specific action; capturing an image that includes, as a subject, the target person performing the specific action; estimating a skeletal model of the target person in the image based on the image captured; setting a plurality of three-dimensional regions around the skeletal model based on positions of a plurality of skeletal points in the skeletal model estimated; identifying, among the plurality of three-dimensional regions set, a three-dimensional region where a skeletal point of a wrist of the target person is located in the specific action; and determining a state of activities of daily living of the target person based on the three-dimensional region identified.

### TECHNICAL FIELD

The present invention relates to a determination method, a determination device, and a determination system.

### BACKGROUND ART

Conventionally, nursing homes provide training (so-called rehabilitation) services so that elderly people can live independently. In the rehabilitation, a staff member at the nursing home who is qualified to produce a training plan visits the home of an elderly person to determine the physical function and the state of activities of daily living (ADL) of the elderly person and to produce a training plan corresponding to the state of the ADL. The rehabilitation is performed according to the training plan which has been produced.

For example, Patent Literature (PTL) 1 discloses an activity information processing device which acquires, in the evaluation of rehabilitation, activity information of a target person who performs a predetermined activity, analyzes the acquired activity information, and displays display information based on an analysis value related to the movement of a specified part.

### CITATION LIST

### Patent Literature

[PTL 1] Japanese Unexamined Patent Application Publication No. 2015-061579

### SUMMARY OF INVENTION

### Technical Problem

However, in the technique disclosed in PTL 1, when a training plan for the rehabilitation is produced, if the state of activities of daily living of the target person is not accurately determined, the rehabilitation of the target person cannot be accurately evaluated.

The present invention provides a determination method, a determination device, and a determination system which can easily and accurately determine the state of activities of daily living of a target person.

### Solution to Problem

A determination method according to an aspect of the present invention is a determination method performed by a computer, and includes: instructing a target person to perform a specific action; capturing an image that includes, as a subject, the target person performing the specific action; estimating a skeletal model of the target person in the image based on the image captured; setting a plurality of three-dimensional regions around the skeletal model based on positions of a plurality of skeletal points in the skeletal model estimated; among identifying, the plurality of three-dimensional regions set, a three-dimensional region where a skeletal point of a wrist of the target person is located in the specific action; and determining a state of activities of daily living of the target person based on the three-dimensional region identified.

A determination device according to an aspect of the present invention includes: an instructor that instructs a target person to perform a specific action; a camera that captures an image which includes, as a subject, the target person performing the specific action; an estimator that estimates a skeletal model of the target person in the image based on the image captured; a setter that sets a plurality of three-dimensional regions around the skeletal model based on positions of a plurality of skeletal points in the skeletal model estimated; an identifier that identifies, among the plurality of three-dimensional regions set, a three-dimensional region which includes a skeletal point of a wrist of the target person in the specific action; and a determiner that determines a state of activities of daily living of the target person based on the three-dimensional region identified.

A determination system according to an aspect of the present invention includes: an information terminal; and a server device that is connected to the information terminal via communication, the information terminal includes: a communicator that communicates with the server device; an instructor that instructs a target person to perform a specific action; and a camera that captures an image which includes, as a subject, the target person performing the specific action, and the server device includes: an estimator that estimates a skeletal model of the target person in the image based on the image captured by the camera; a setter that sets a plurality of three-dimensional regions around the skeletal model based on positions of a plurality of skeletal points in the skeletal model estimated; an identifier that identifies, among the plurality of three-dimensional regions set, a three-dimensional region which includes a skeletal point of a wrist of the target person in the specific action; and a determiner that determines a state of activities of daily living of the target person based on the three-dimensional region identified.

### Advantageous Effects of Invention

According to the present invention, a determination method, a determination device, and a determination system which can easily and accurately determine the state of activities of daily living of a target person are realized.

### BRIEF DESCRIPTION OF DRAWINGS

FIG. 1 is a block diagram showing an example of the functional configuration of a determination system according to an embodiment.

FIG. 2 is a flowchart showing a first example of the operation of the determination system according to the embodiment.

FIG. 3 is a flowchart showing a second example of the operation of the determination system according to the embodiment.

FIG. 4 is a diagram conceptually showing estimation of a two-dimensional skeletal model of a target person.

FIG. 5 is a diagram conceptually showing estimation of a three-dimensional skeletal model.

FIG. 6 is a diagram conceptually showing setting of a plurality of three-dimensional regions.

FIG. 7 is a diagram conceptually showing identification of a three-dimensional region where a wrist is located.

FIG. 8 is a diagram showing an example of a database.

FIG. 9 is a diagram showing an example of presentation information.

FIG. 10 is a diagram showing an example of the presentation information.

FIG. 11 is a diagram showing an example of the presentation information.

FIG. 12 is a diagram showing another example of the presentation information.

FIG. 13 is a diagram showing another example of the presentation information.

### DESCRIPTION OF EMBODIMENTS

Embodiments will be specifically described below with reference to drawings. Each of the embodiments described below indicates a comprehensive or specific example. Numerical values, shapes, materials, constituent elements, the arrangement and connection of the constituent elements, steps, the order of the steps, and the like shown in the following embodiments are examples, and are not intended to limit the present invention. Among the constituent elements in the following embodiments, constituent elements which are not recited in the independent claims are described as optional constituent elements.

The drawings are schematic views and are not exactly shown. In the drawings, substantially the same configurations are identified with the same reference signs, and repeated description may be omitted or simplified.

### Embodiment

### 1. CONFIGURATION

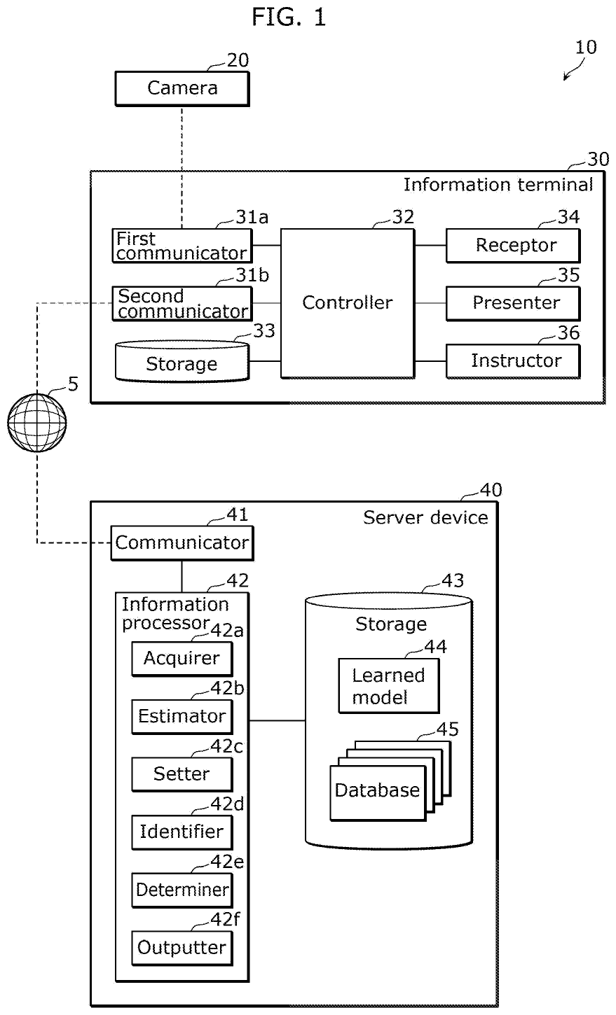

The configuration of a determination system according to an embodiment will first be described. FIG. 1 is a block diagram showing an example of the functional configuration of the determination system according to the embodiment.

Determination system 10 sets a plurality of three-dimensional regions around a skeletal model which is estimated based on an image of a target person who performs a specific action, identifies, among the set three-dimensional regions, a three-dimensional region including the skeletal point of a wrist of the target person in the specific action, and determines the state of activities of daily living of the target person based on the identified three-dimensional region. A determination method will be described later.

The target person is, for example, a person whose physical function, that is, the ability to move the body, is impaired due to illness, trauma, aging, or disability. Examples of a user include a physical therapist, an occupational therapist, a nurse, and a rehabilitation specialist.

As shown in FIG. 1, determination system 10 includes, for example, camera 20, information terminal 30, and server device 40.

### [Camera]

Camera 20 captures an image (for example, moving images which include a plurality of images) which includes, as a subject, the target person who performs the specific action. Camera 20 may be a camera which uses a complementary metal oxide semiconductor (CMOS) image sensor or may be a camera which uses a charge coupled device (CCD) image sensor. Although in the example of FIG. 1, camera 20 is a camera which is connected to information terminal 30 by communication, camera 20 may be an external camera which is attached to information terminal 30 or may be a camera which is installed in information terminal 30.

### [Information Terminal]

Information terminal 30 instructs the target person to perform the specific action, acquires the image (more specifically, image data or image information) of the target person captured by camera 20, and transmits the acquired image to server device 40. Although information terminal 30 is, for example, a portable computer device such as a smartphone or a tablet terminal used by the user, information terminal 30 may be a stationary computer device such as a personal computer. Specifically, information terminal 30 includes first communicator 31a, second communicator 31b, controller 32, storage 33, receptor 34, presenter 35, and instructor 36.

First communicator 31a is a communication circuit (that is, a communication module) with which information terminal 30 communicates with camera 20 via a local communication network. First communicator 31a is, for example, a wireless communication circuit which performs wireless communication but first communicator 31a may be a wired communication circuit which performs wired communication. The communication standard of communication performed by first communicator 31a is not particularly limited. For example, first communicator 31a may communicate with camera 20 by Wi-Fi (registered trademark) or the like via a router (not shown) or may directly communicate with camera 20 by Bluetooth (registered trademark) or the like.

Second communicator 31b is a communication circuit (that is, a communication module) with which information terminal 30 communicates with server device 40 via wide area communication network 5 such as the Internet. Second communicator 31b is, for example, a wireless communication circuit which performs wireless communication but second communicator 31b may be a wired communication circuit which performs wired communication. The communication standard of communication performed by second communicator 31b is not particularly limited.

Controller 32 performs various types of information processing on information terminal 30 based on an operation input received by receptor 34. Controller 32 is realized by, for example, a microcomputer but may be realized by a processor.

Storage 33 is a storage device in which dedicated application programs and the like to be executed by controller 32 are stored. Storage 33 is realized by, for example, a semiconductor memory.

Receptor 34 is an input interface which receives an operation input performed by the user of information terminal 30 (for example, a rehabilitation specialist). For example, receptor 34 receives an input operation performed by the user for transmitting, to server device 40, conditions for assigning weights in a determination performed by determiner 42e, conditions for extraction of the result of the determination, or conditions for a method for presentation to presenter 35 and an instruction to start or complete a measurement. Specifically, receptor 34 is realized by a touch panel display or the like. For example, when a touch panel display is installed in receptor 34, the touch panel display functions as presenter 35 and receptor 34. Receptor 34 is not limited to a touch panel display, and may be, for example, a keyboard, a pointing device (such as a touch pen or a mouse), or a hardware pointer. When receptor 34 receives an input of voice, receptor 34 may be a microphone. When receptor 34 receives an input of a gesture, receptor 34 may be a camera.

For example, presenter 35 presents, to the user, the result of the determination of the state of activities of daily living. Presenter 35 also presents, to the user, information about the state of activities of daily living of the target person extracted based on an instruction of the user. Instructor 36 is, for example, at least one of a display panel such as a liquid crystal panel or an organic electro luminescence (EL) panel, a speaker, or earphones. For example, when an instruction is provided by voice and video, presenter 35 may be a display panel and a speaker or earphones or may be a display panel, a speaker and earphones.

Instructor 36 instructs the target person to perform the specific action. Instructor 36 may provide an instruction to the target person by at least one of voice, characters, or video. Instructor 36 is, for example, at least one of a display panel such as a liquid crystal panel or an organic EL panel, a speaker, or earphones. For example, when an instruction is provided by voice and video, instructor 36 may be a display panel and a speaker or earphones or may be a display panel, a speaker and earphones.

Instructor 36 may function as presenter 35 depending on the form of an instruction or presenter 35 may function as instructor 36. In other words, instructor 36 may be integral with presenter 35.

### [Server Device]

Server device 40 acquires the image transmitted from information terminal 30, estimates a skeletal model in the acquired image, and determines the state of activities of daily living of the target person based on the estimated skeletal model. Server device 40 includes communicator 41, information processor 42, and storage 43.

Communicator 41 is a communication circuit (that is, a communication module) with which server device 40 communicates with information terminal 30. Communicator 41 may include a communication circuit (communication module) for communication via wide area communication network 5 and a communication circuit (communication module) for communication via a local communication network. Communicator 41 is, for example, a wireless communication circuit which performs wireless communication. The communication standard of communication performed by communicator 41 is not particularly limited.

Information processor 42 performs various types of information processing on server device 40. Information processor 42 is realized by, for example, a microcomputer but may be realized by a processor. For example, a microcomputer, a processor, or the like of information processor 42 executes a computer program stored in storage 43, and thus the function of information processor 42 is realized. Specifically, information processor 42 includes acquirer 42a, estimator 42b, identifier 42d, determiner 42e, and outputter 42f.

Acquirer 42a acquires the image (for example, moving images which include a plurality of images) transmitted from information terminal 30 and the operation input performed by the user and received by receptor 34.

Estimator 42b estimates the skeletal model of the target person in the image based on the image acquired by acquirer 42a. More specifically, estimator 42b estimates, based on moving images which include a plurality of images, a skeletal model in each of the images included in the moving images. For example, estimator 42b estimates a two-dimensional skeletal model of the target person based on the image, and estimates a three-dimensional skeletal model of the target person based on the estimated two-dimensional skeletal model using learned model 44 which is a learned machine learning model.

Setter 42c sets, based on the positions of a plurality of skeletal points in the skeletal model estimated by estimator 42b, a plurality of three-dimensional regions around the skeletal model. More specifically, for example, setter 42c sets the three-dimensional regions based on the three-dimensional skeletal model. For example, setter 42c sets the three-dimensional regions around the skeletal model with one of the skeletal points in the skeletal model used as a base point. Since the two-dimensional skeletal model, the estimation of the three-dimensional skeletal model, and the setting of the three-dimensional regions will be described in detail in [First example] of [2. Operation], the description is omitted here.

Identifier 42d identifies, among the three-dimensional regions set by setter 42c, a three-dimensional region where the skeletal point of a wrist of the target person is located in the specific action.

Determiner 42e determines the state of activities of daily living of the target person based on the three-dimensional region identified by identifier 42d. For example, determiner 42e determines, based on database 45 in which the specific action, a three-dimensional region where the wrist is located in the specific action, and an activity of daily living corresponding to the specific action are stored in association with each other, whether the three-dimensional region identified by identifier 42d matches the three-dimensional region stored in database 45, and thereby determines the state of activities of daily living of the target person.

Outputter 42f outputs, for example, at least one of the result of the determination of the state of activities of daily living of the target person or information about the state of activities of daily living of the target person. Outputter 42f may further output the three-dimensional skeletal model in the moving images of the target person, a characteristic amount (for example, data of the physical function such as a joint movable range) used for the result of the determination of the state of activities of daily living, the result of the determination of the physical function of the target person, a rehabilitation training plan, or the like.

Storage 43 is a storage device in which image data acquired by acquirer 42a is accumulated. In storage 43, computer programs executed by information processor 42 and the like are also stored. For example, in storage 43, database 45 in which the specific action, the three-dimensional region where the wrist is located in the specific action, and the activity of daily living corresponding to the specific action are stored in association with each other and the learned machine learning model (learned model 44) are stored. Specifically, storage 43 is realized by a semiconductor memory, a hard disk drive (HDD), or the like.

Although in the example of FIG. 1, determination system 10 includes a plurality of devices, determination system 10 may be a single device.

### 2. OPERATION

The operation of determination system 10 will then be specifically described with reference to drawings.

### First Example

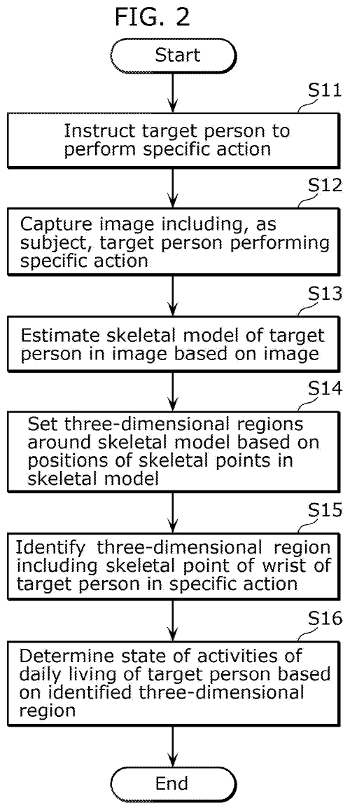

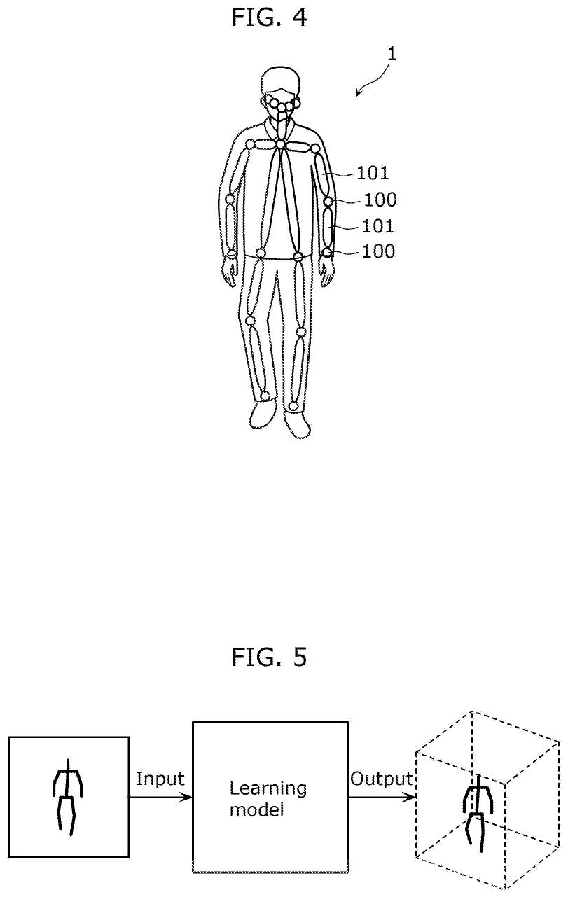

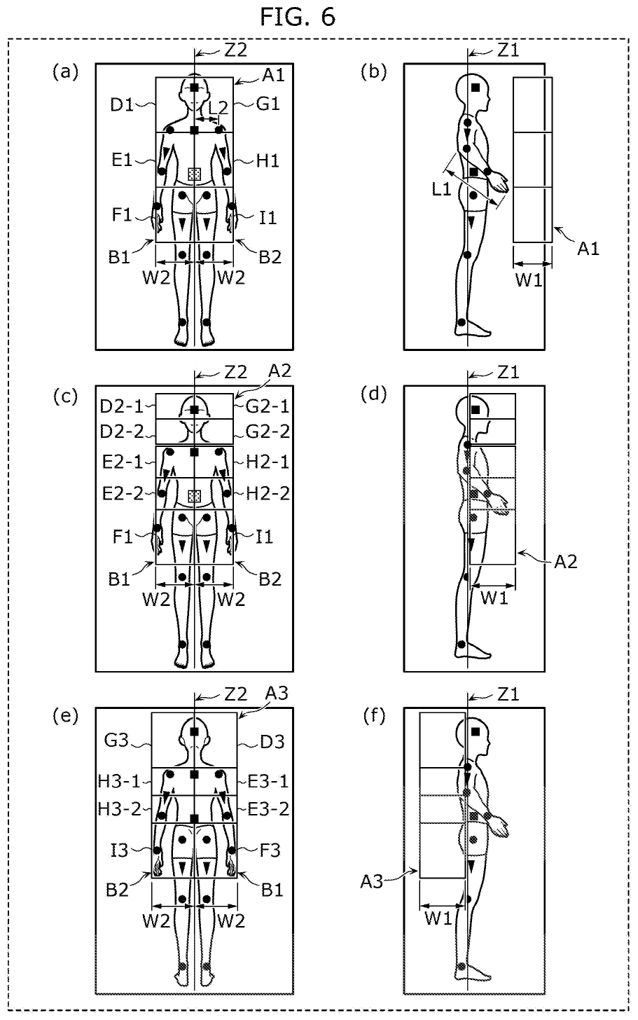

A first example of the operation will first be described with reference to FIG. 2. FIG. 2 is a flowchart showing the first example of the operation of determination system 10 according to the embodiment. FIG. 4 is a diagram conceptually showing setting of a plurality of three-dimensional regions. FIG. 5 is a diagram conceptually showing estimation of a two-dimensional skeletal model of the target person. FIG. 6 is a diagram conceptually showing estimation of a three-dimensional skeletal model.

Although not shown in the figure, when receptor 34 receives an instruction to start the operation, determination system 10 acquires an image captured by camera 20, and identifies the target person in the acquired image. For the identification of the target person in the image, a known image analysis technique is used.

Then, when the target person is identified by determination system 10, instructor 36 instructs the target person to perform the specific action (S11).

Then, camera 20 captures an image which includes, as a subject, the target person performing the specific action (S12), and transmits the captured image (hereinafter also referred to as image data) to information terminal 30 (not shown). In step S12, camera 20 may capture moving images which include a plurality of images.

Then, information terminal 30 acquires the image data transmitted from camera 20 via first communicator 31a (not shown), and transmits the acquired data to server device 40 via second communicator 31b (not shown). Here, information terminal 30 may anonymize the image data and transmit it to server device 40. In this way, the privacy data of the target person is protected.

The, estimator 42b of server device 40 estimates the skeletal model of the target person in the image based on the image (image data) acquired by acquirer 42a (S13). When acquirer 42a acquires moving images including a plurality of images, estimator 42b may estimate a skeletal model in each of the images included in the moving images based on the acquired moving images.

For example, in step S13, estimator 42b may estimate the two-dimensional skeletal model of the target person based on the image, and estimate the three-dimensional coordinate data (so-called three-dimensional skeletal model) of the target person based on the estimated two-dimensional skeletal model using learned model 44 that is a learned machine learning model.

FIG. 4 is a diagram conceptually showing estimation of the two-dimensional skeletal model of the target person. As shown in FIG. 4, the two-dimensional skeletal model is a model in which the positions (circles in the figure) of joints 100 of target person 1 shown in the image are connected by links (lines in the figure). For the estimation of the two-dimensional skeletal model, an existing posture and skeletal estimation algorithm is used.

FIG. 5 is a diagram conceptually showing estimation of the three-dimensional skeletal model. Learned model 44 (learning model in the figure) is an identifier which is previously constructed by machine learning in which a two-dimensional skeletal model with known three-dimensional coordinate data of joints is used as learning data and the three-dimensional coordinate data is used as teacher data. Learned model 44 uses the two-dimensional skeletal model as an input to be able to output its three-dimensional coordinate data, that is, the three-dimensional skeletal model.

For example, in step S13, estimator 42b may estimate three-dimensional coordinate data (three-dimensional skeletal model) based on the image acquired by acquirer 42a. In this case, for example, a learned model which shows a relationship between the image of the target person and the three-dimensional coordinate data may be used.

Then, setter 42c sets, based on the positions of a plurality of skeletal points in the skeletal model estimated by estimator 42b in step S13, a plurality of three-dimensional regions around the skeletal model. More specifically, for example, setter 42c sets the three-dimensional regions based on the three-dimensional skeletal model. For example, setter 42c sets the three-dimensional regions around the skeletal model with one of the skeletal points in the skeletal model used as the base point. The setting of the three-dimensional regions will be specifically described below.

FIG. 6 is a diagram conceptually showing setting of a plurality of three-dimensional regions. A description will first be given with reference to parts (b), (d), and (f) in FIG. 6. As shown in parts (b), (d), and (f) in FIG. 6, in a side view of the target person, the three-dimensional regions are included in any one of back surface region A3 (see part (f) in FIG. 6) on a back surface side of the target person, front surface region A2 (see part (d) in FIG. 6) on a front surface side of the target person, or forward region A1 (see part (b) in FIG. 6), back surface region A3 and front surface region A2 are provided adjacent to each other through first reference axis Z1 in a longitudinal direction that extends from the head of the target person to the legs of the target person and passes through the base point, and forward region A1 is provided on a forward side of the target person to be adjacent to the front surface region. As shown in parts (a), (c), and (e) in FIG. 6, in a front view of the target person, each of back surface region A3, front surface region A2, and forward region A1 includes left side region B2 and right side region B1 of the target person that are provided adjacent to each other through second reference axis Z2 in the longitudinal direction which passes through the base point, and each of left side region B2 and right side region B1 includes a predetermined number of regions divided in the longitudinal direction from the head of the target person to the legs of the target person. For example, in part (a) in FIG. 6, each of left side region B2 and right side region B1 in forward region A1 includes three regions divided in a lateral direction orthogonal to the longitudinal direction from the head of the target person to the legs of the target person. Although as shown in FIG. 6, the predetermined number of regions included in right side region B1 is the same as the predetermined number of regions included in left side region B2, a different number of regions may be included in each of back surface region A3, front surface region A2, and forward region A1.

For example, in first reference axis Z1, the skeletal point of a neck of the target person and the skeletal point of a waist of the target person may be set as the base point, and in second reference axis Z2, the skeletal point of the neck of the target person and the skeletal point of an elbow of the target person may be set as the base point. In this case, for example, as shown in parts (b), (d), and (f) in FIG. 6, setter 42c may set, in the side view of the target person, first distance L1 from the skeletal point of the elbow of the target person to a tip of a hand of the target person as width W1 of each of back surface region A3, front surface region A2, and forward region A1, and for example, as shown in parts (a), (c), and (e) in FIG. 6, setter 42c may set, in the front view of the target person, a distance twice second distance L2 from the skeletal point of the neck of the target person to the skeletal point of a shoulder of the target person as width W2 of each of left side region B2 and right side region B1. The setting of the base points and the widths described above is an example, and the present invention is not limited to this example.

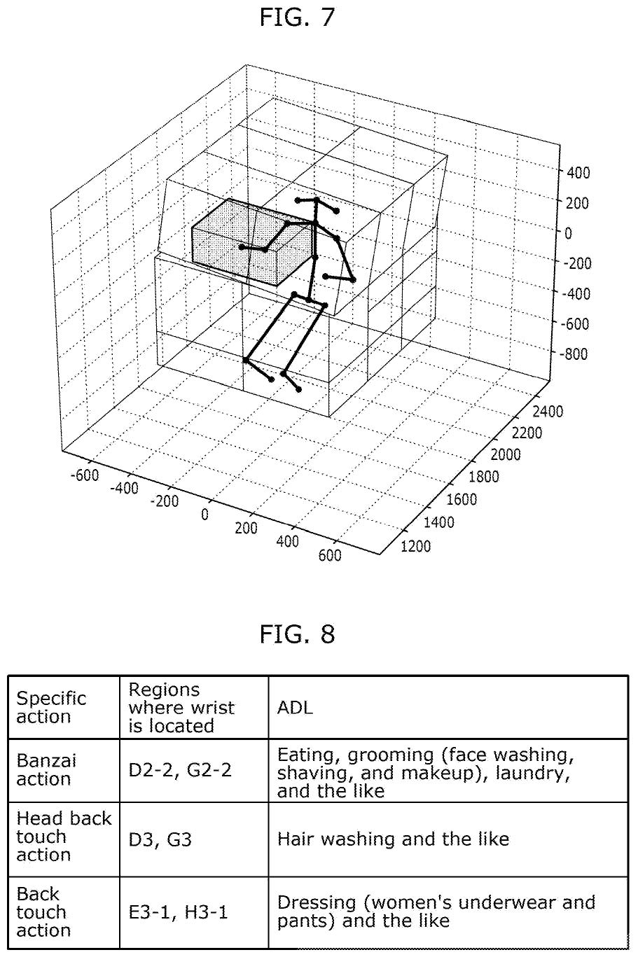

A description will be given with reference back to FIG. 2. When in step S14, the three-dimensional regions are set around the skeletal model by setter 42c, identifier 42d identifies, among the three-dimensional regions set by setter 42c, a three-dimensional region where the skeletal point of the wrist of the target person is located in the specific action (S15). FIG. 7 is a diagram conceptually showing identification of the three-dimensional region where the wrist is located, Identifier 42d identifies, based on the three-dimensional coordinate data (so-called three-dimensional skeletal model) of the target person in the image, in which one of the three-dimensional regions the coordinates of the skeletal point of the wrist of the target person are located (that is, are included). The identified three-dimensional region is a shaded region shown in FIG. 7.

Then, determiner 42e determines the state of activities of daily living of the target person based on the three-dimensional region identified by identifier 42d in step S15 (S16). For example, determiner 42e may reference database 45 in which the specific action, the three-dimensional region where the wrist is located in the specific action, and the activity of daily living corresponding to the specific action are stored in association with each other, and determine the state of activities of daily living of the target person by determining whether the three-dimensional region identified by identifier 42d matches the three-dimensional region stored in database 45 in association with the specific action.

FIG. 8 is a diagram showing an example of database 45. As shown in FIG. 8, in database 45, the specific actions, the three-dimensional regions where the wrists of the target person are located in the specific actions, and the activities of daily living (ADL) are stored in association with each other. For example, when the specific action is a banzai action, and thus the three-dimensional regions where the wrists of the target person are located in the specific action are D2-2 (region where the right wrist is located) and G2-2 (region where the left wrist is located) shown in FIG. 6, it is determined that activities of daily living such as eating, grooming (face washing, shaving, and makeup), and laundry can be performed.

When the processing in steps S11 to S16 is assumed to be one-loop processing, determination system 10 may perform the one-loop processing every time the target person performs each of a plurality of specific actions. Alternatively, the processing in steps S11 and S12 may be performed for each of a plurality of specific actions, and after the target person completes all the specific actions, the processing in steps S13 to S16 may be performed for each of the specific actions.

As described above, in determination system 10 according to the present embodiment, the skeletal model in the image which includes, as the subject, the target person performing the specific action is estimated, a plurality of three-dimensional regions are set around the estimated skeletal model, in which one of the three-dimensional regions the wrist of the target person is located is identified, and thus it is possible to easily and accurately determine the state of activities of daily living of the target person.

### Variation 1 of First Example

Although in the first example, the specific action is not selected according to the physical function of the target person when an instruction is provided to the target person to perform the specific action, in a variation of the first example, before the provision of an instruction for the specific action, the action which the target person is caused to perform may be selected.

For example, before step S11 in FIG. 2, an instruction may be provided to the target person to perform an action of standing up from a sitting posture. Here, determination system 10 may determine, based on an image of the target person captured by camera 20, whether the target person can perform the action of standing up or may make the determination by an instruction of the user. The determination may be performed by determiner 42e. The determination based on the image may be performed, for example, by estimating the skeletal model of the target person in the image. For example, the instruction of the user may be a gesture or voice or may be input by operating a touch panel or a button of a remote controller. For example, when the target person cannot perform the action of standing up, the gesture may be waving one hand from side to side, shaking the head from side to side, or crossing both arms to form a cross, and when the target person can perform the action of standing up, the gesture may be a nod of the head, a thumbs up, or making a circle with both hands. The voice may be, for example, a short utterance of “no” or “yes”.

As described above, the specific action is selected according to the physical function of the target person, and thus it is possible to efficiently and accurately determine the state of activities of daily living of the target person.

### Variation 2 of First Example

In the first example and Variation 1 of the first example, determination system 10 sets a plurality of three-dimensional regions based on the three-dimensional skeletal model of the target person performing the specific action, identifies the three-dimensional region where the wrist of the target person is located in the specific action, and thereby determines the state of activities of daily living of the target person. In Variation 2 of the first example, the state of activities of daily living of the target person is determined by further determining whether an action accompanied by a movement of fingers of the target person (for example, an action of opening and closing a hand (clasping and unclasping a hand) or an action of opposing fingers (OK sign)) can be performed.

For example, when receptor 34 of information terminal 30 receives an instruction to determine whether an action accompanied by a movement of fingers can be performed, controller 32 causes instructor 36 to instruct the target person to perform the action accompanied by the movement of fingers.

When information terminal 30 acquires an image which is captured by camera 20 and includes, as a subject, the target person who performs the action accompanied by the movement of fingers, information terminal 30 transmits, to server device 40, the instruction received by receptor 34 and the image (specifically, image data) captured by camera 20.

When determiner 42e of server device 40 uses, for example, another learned model (not shown) different from learned model 44 to identify an action of clasping and unclasping a hand in the image, determiner 42e may determine that the target person can perform the action of opening and closing a hand. Determiner 42e may determine whether the target person can perform the action of opposing fingers by using another learned model to identify whether a tip of the index finger is attached to a tip of the thumb in the image and the shape and size of a space between the index finger and the thumb.

As described above, whether an action accompanied by a movement of fingers of the target person can be performed is determined, and thus it is possible to determine, for example, whether the target person can grasp an object, with the result that it is possible to more accurately determine the state of activities of daily living of the target person.

### Variation 3 of First Example

In Variation 3 of the first example, based on the skeletal model estimated by estimator 42b, a characteristic amount which indicates the characteristic of the movement of the skeleton of the target person in the specific action is derived, and the physical function which is the ability of the target person to perform a physical activity is determined based on the characteristic amount.

A description will be given with reference back to FIG. 4. For example, determiner 42e derives, based on the skeletal model estimated by estimator 42b, the positions of two non-articular parts 101 of target person 1 which are connected via predetermined joint 100, and derives, based on a straight line connecting the positions of two non-articular parts 101 derived, as the characteristic amount, a Joint angle (not shown) related to at least one of flexion, extension, abduction, adduction, external rotation, or internal rotation of predetermined joint 100. For example, a joint angle related to the flexion of an elbow joint is derived based on three-dimensional coordinate data (three-dimensional skeletal model) estimated based on a two-dimensional skeletal model. For example, determiner 42e may determine the physical function of the target person based on a database (not shown) in which the range of the joint angle related to the flexion of the elbow joint in the specific action and the result of the determination of the physical function are stored in association with each other. In the database, not only the joint angle but also the following characteristic amounts may likewise be stored in association with the result of the determination of the physical function.

For example, determiner 42e may derive a distance between predetermined joint 100 and a terminal part in the specific action, a variation width of the position of predetermined joint 100, and the like to determine whether the values thereof are equal to or greater than threshold values or whether the values fall in predetermined ranges.

For example, determiner 42e may derive a variation in the position of predetermined joint 100 or a terminal part (for example, a tip of a hand) and a variation width to determine whether the sway of the body of target person 1 occurs when the specific action is performed.

As described above, based on the skeletal model of the target person, the characteristic amount indicating the characteristic of the movement of the skeleton of the target person in the specific action is derived, and based on the derived characteristic amount, the physical function of the target person is determined, with the result that it is possible to grasp not only the state of activities of daily living but also the physical function such as muscle strength. In this way, it is possible to provide, based on the physical function such as muscle strength, a training plan necessary for maintaining or enhancing the physical function to, for example, the target person who has no problem with activities of daily living.

### Second Example

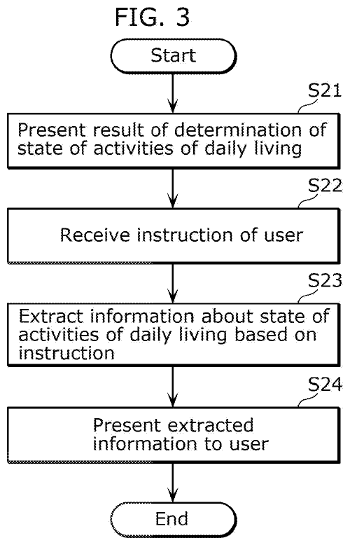

A second example of the operation will then be described with reference to FIG. 3. FIG. 3 is a flowchart showing the second example of the operation of determination system 10 according to the embodiment. In the second example, an example is described where information about the state of activities of daily living which is extracted based on an instruction of the user from the state of activities of daily living of the target person determined in the first example is presented.

Although not shown in the figure, when the processing flow shown in FIG. 2 is completed, determiner 42e outputs the state of activities of daily living of the target person (hereinafter also referred to as the result of the determination) to outputter 42f. Outputter 42f outputs the result of the determination acquired to information terminal 30 via communicator 41. Here, the result of the determination which is output may be anonymized by information processor 42.

Then, when information terminal 30 acquires the result of the determination from server device 40, presenter 35 presents the result of the determination of the state of activities of daily living which is acquired (S21). In step S21, when the target person performs a plurality of specific actions, the result of the determination of the state of activities of daily living which is associated with each of the specific actions may be presented or only the result of the determination which is not satisfactory may be presented. These results of the determination may be presented in order of inferior results.

Then, receptor 34 receives an instruction from the user (S22). The instruction of the user may be specification of extraction conditions for extracting desired information under predetermined conditions from the result of the determination, may be specification of a presentation method of the result of the determination, or may be specification of the extraction conditions and the presentation method. The desired information may be, for example, a three-dimensional skeletal model in an image which includes, as a subject, the target person performing the specific action, a three-dimensional skeletal model in an exemplary image, the state of the physical function, or the like. Examples of the presentation method include presentation of only image information including characters, presentation of image information and voice information, and the like.

Then, information terminal 30 transmits, to server device 40, the instruction of the user received by receptor 34 in step S22 (not shown). When determiner 42e of server device 40 acquires the instruction of the user from information terminal 30, determiner 42e extracts information about the state of activities of daily living based on the instruction of the user (S23). For example, when the instruction of the user is specification of extraction conditions for assigning weights to activities of daily living about transfers, the result of the determination of the state of activities of daily living about transfers among activities of daily living corresponding to the specific actions is preferentially extracted. Outputter 42f of server device 40 outputs, to information terminal 30, the information (hereinafter also referred to as the extracted information or the result of the extraction) about activities of daily living which is extracted by determiner 42e in step S23 (not shown). For example, the information about the state of activities of daily living includes at least one of a three-dimensional skeletal model of the target person performing the specific action, the result of the determination of the physical function of the target person, or a detail of training to be proposed to the target person. The information about the state of activities of daily living includes the physical function of the target person, and the physical function of the target person is determined based on the state of at least one of an action of opening and closing a hand (clasping and unclasping a hand) of the target person or an action of opposing fingers (OK sign) of the target person.

Then, when information terminal 30 acquires the result of the extraction from server device 40, presenter 35 presents, to the user, the information about the state of activities of daily living which is extracted in step S23 (S24).

Although in the second example, after the presentation of the result of the determination of the state of daily living of the target person, the information about the state of activities of daily living which is extracted from the result of the determination under predetermined conditions by the instruction of the user is presented, the user may input an instruction for extraction conditions or the like before the presentation of the result of the determination. Here, determination system 10 may notify the user of, for example, the completion of the determination before the presentation of the result of the determination. In this way, it is possible to extract information desired by the user from the result of the determination and present it to the user.

### Variation 1 of Second Example

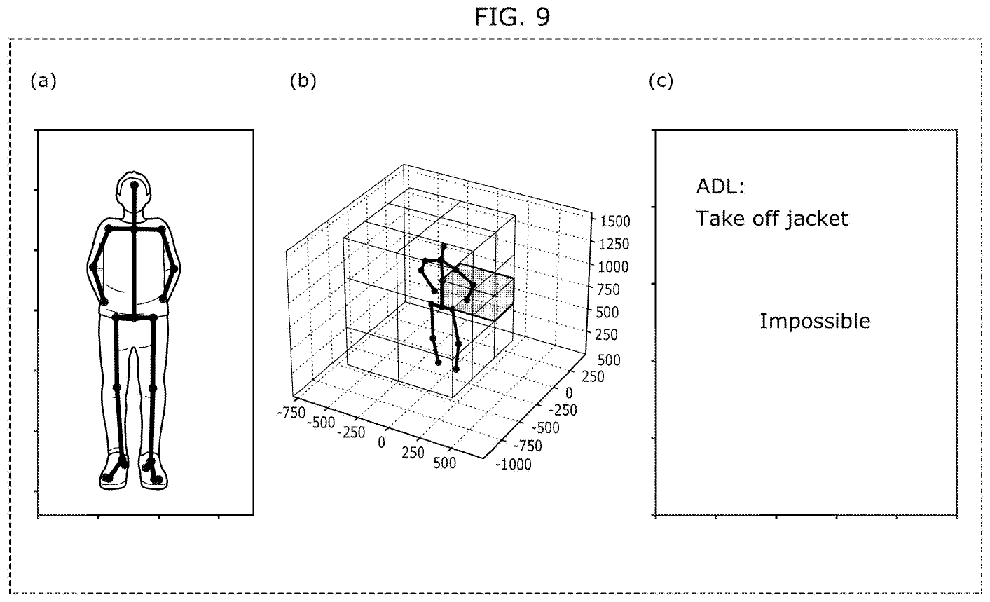

Variation 1 of the second example will then be described with reference to FIGS. 9, 10, and 11. FIG. 9 is a diagram showing an example of the determination of the state of activities of daily living in a back touch action. FIGS. 9 to 11 are diagrams showing examples of presentation information. In the following description, the details described with reference to FIGS. 2 and 3 will be omitted or simplified.

Although in the second example, the result of the determination of the state of activities of daily living is presented to the user, in the variation of the second example, while the determination of activities of daily living is being performed, the result of the determination and the information about the state of activities of daily living are presented to the user.

For example, when receptor 34 of information terminal 30 receives an instruction to make a presentation in parallel with the determination, information terminal 30 transmits the instruction to server device 40.

When server device 40 acquires the instruction, information processor 42 outputs, to information terminal 30, presentation information which presenter 35 is caused to present.

When information terminal 30 acquires the presentation information, presenter 35 presents the presentation information, and instructor 36 instructs the target person to perform the specific action (step S11 in FIG. 2). For example, the instruction may be provided by outputting a voice such as “raise your hands with the hands tied behind your back”.

In each of FIGS. 9 to 11, part (a) shows a two-dimensional skeletal model in an image (here, moving images) captured by camera 20, part (b) shows a three-dimensional skeletal model and a plurality of three-dimensional regions, and part (c) shows an activity of daily living (ADL) corresponding to the specific action and the result of the determination thereof.

Camera 20 captures the image (here, the moving images) which includes, as a subject, the target person performing the specific action (S12 in FIG. 2), and estimator 42b estimates the skeletal model of the target person based on the captured moving images (S13). In Variation 1 of the second example, the processing in step S23 in FIG. 3 is performed in parallel with the processing in step S13. For example, when the two-dimensional skeletal model and the three-dimensional skeletal model are estimated in step S13, these skeletal models are presented to presenter 35.

Then, setter 42c sets, based on the positions of a plurality of skeletal points (circles in the figure) in the estimated skeletal model, a plurality of three-dimensional regions around the skeletal model (S14 in FIG. 2). In Variation 1 of the second example, the processing in step S23 in FIG. 3 is performed in parallel with the processing in step S14. For example, when the three-dimensional regions are set in step S14, as shown in part (b) in each of FIGS. 9 to 11, the image in which the three-dimensional regions are displayed around the three-dimensional skeletal model is presented to presenter 35.

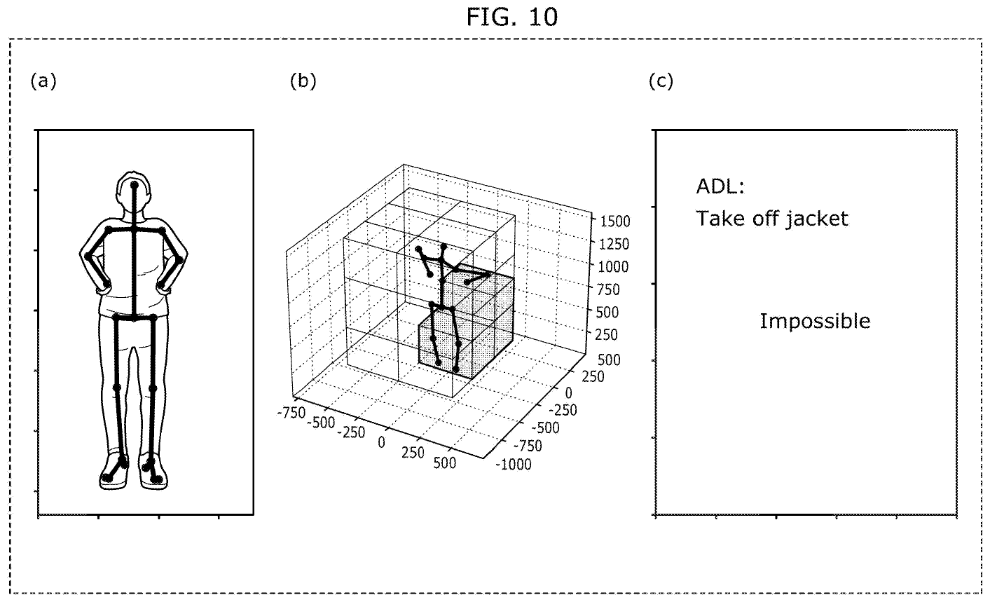

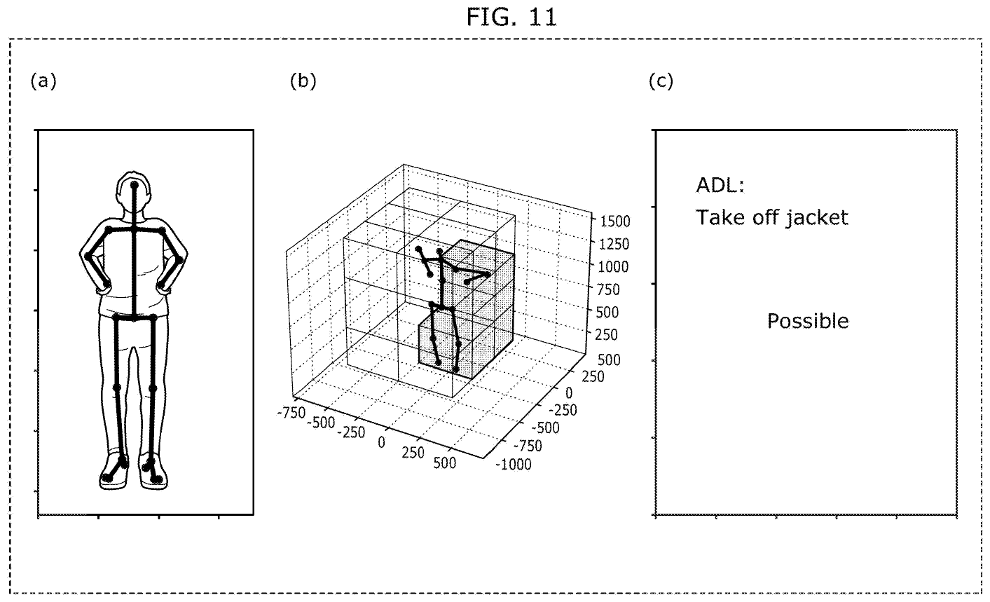

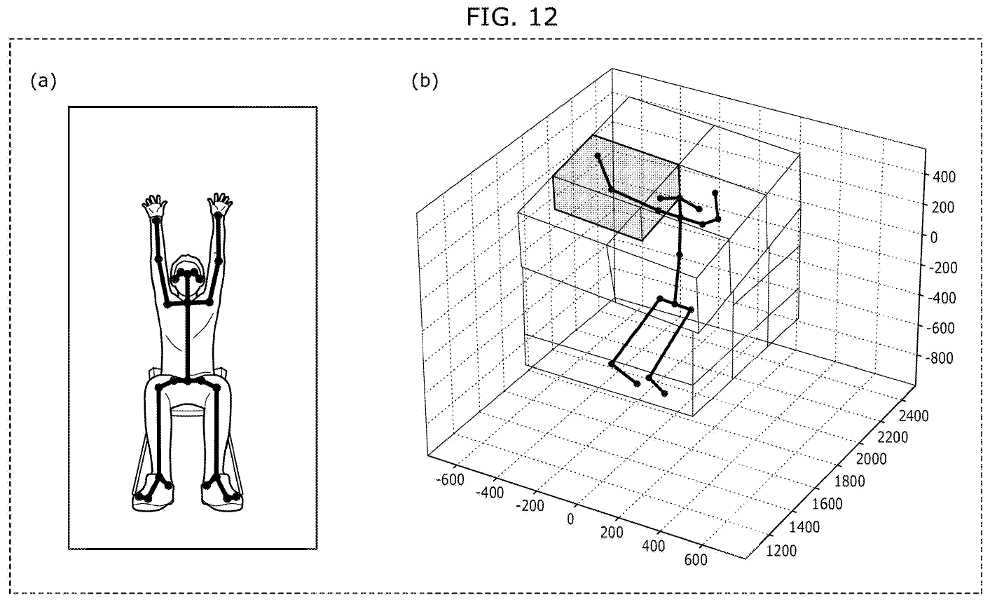

Then, identifier 42d identifies the three-dimensional region where the skeletal point of the wrist of the target person is located in the specific action amount the three-dimensional regions set by setter 42c (S15 in FIG. 2), and determiner 42e determines the state of activities of daily living of the target person based on the three-dimensional region identified by identifier 42d in step S15 (S16 in FIG. 2). In Variation 1 of the second example, the processing in steps S21 and S23 in FIG. 3 is performed in parallel with the processing in steps S15 and S16. For example, when the three-dimensional regions are set in step S15, as shown in part (b) in FIG. 9, an image in which the three-dimensional region where the wrist of the target person is located is marked up among the three-dimensional regions is presented to presenter 35. As shown in part (b) in each of FIGS. 10 and 11, the three-dimensional regions through which the wrist has passed may also be marked up so that the movement trajectory of the position of the wrist of the target person can be seen. As shown in part (b) in each of FIGS. 9 to 11, in terms of visibility, only the three-dimensional regions where the position of one wrist is located may be marked up or the three-dimensional regions where the positions of the wrists are located may be marked up. For example, when the state of activities of daily living of the target person is determined in step S16, as shown in part (c) in each of FIGS. 9 to 11, the activity of daily living (ADL) associated with the specific action and the result of the determination of the state of the activity of daily living are presented. In part (c) in each of FIGS. 9 and 10, when the specific action is the back touch action, the wrists of the target person are not located in the regions (three-dimensional regions E3-1 and H3-1) (see FIGS. 6 and 8) where the wrists are located in the specific action, and thus it is determined that it is impossible to enter the state of activities of daily living related to dressing such as taking off a jacket, with the result that the result of the determination is displayed on presenter 35. On the other hand, in part (c) in FIG. 11, the wrists of the target person are located in the regions where the wrists are located in the specific action, and thus it is determined that it is possible to enter the state of activities of daily living related to dressing such as taking off a jacket, with the result that the result of the determination is displayed on presenter 35.

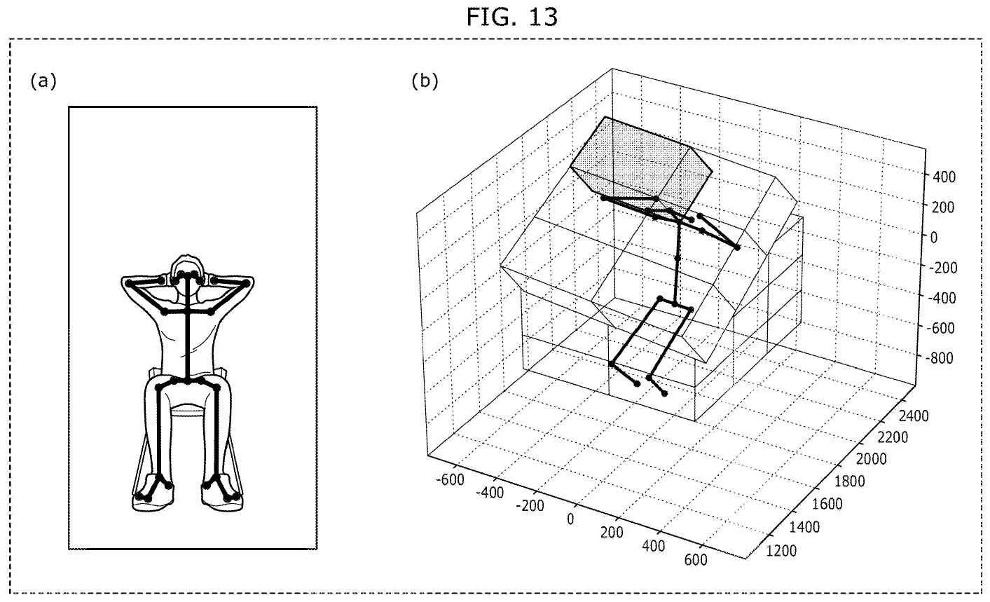

Presenter 35 may output the result of the determination in the presentation information described above by voice. FIGS. 12 and 13 are diagrams showing other examples of the presentation information. In each of FIGS. 12 and 13, part (a) shows a two-dimensional skeletal model in an image (here, moving images) captured by camera 20, and part (b) shows a three-dimensional skeletal model and a plurality of three-dimensional regions.

In FIG. 12, the specific action is a banzai action, the wrists of the target person are located in the regions (three-dimensional regions D2-2 and G2-2) (see FIGS. 6 and 8) where the wrists are located in the specific action, and thus it is determined that it is possible to enter the state of activities of daily living related to eating, grooming (face washing, shaving, and makeup), laundry, and the like, with the result that the result of the determination is presented to the user by voice.

In FIG. 13, the specific action is a head back touch action, the wrists of the target person are located in the regions (three-dimensional regions D3 and G3) (see FIGS. 6 and 8) where the wrists are located in the specific action, and thus it is determined that it is possible to enter the state of activities of daily living related to hair washing and the like, with the result that the result of the determination is presented to the user by voice.

As shown in FIGS. 12 and 13, regions on the upper side (that is, the head side) relative to the skeletal point of the neck among the three-dimensional regions are set according to the direction of the face and the inclination of the neck. In this way, for example, it is possible to determine the state of activities of daily living including whether a vicarious activity is performed.

### Variation 2 of Second Example

In Variation 2 of the second example, in addition to the result of the determination of the state of activities of daily living and the information about activities of daily living, a training plan for rehabilitation is produced and is presented to the user. Specifically, information processor 42 of server device 40 produces a training plan for rehabilitation based on the result of the determination of the state of activities of daily living of the target person. Here, for example, information processor 42 may produce, in addition to the result of the determination of the state of activities of daily living, a training plan for rehabilitation based on the result of the determination of the physical function of the target person.

For example, when an activity of daily living is determined to be impossible among results of the determination based on a plurality of specific actions, information processor 42 may produce a training plan for allowing the activity of daily living described above. For example, even when all the results of the determination based on a plurality of specific actions are determined to be possible, information processor 42 may select an activity of daily living the result of which is inferior to the other results of the determination and produce a training plan for enhancing or maintaining the physical function so that the target person can more smoothly perform the activity of daily living. For example, in addition to the result of the determination described above, based on the result of the determination of the physical function of the target person, information processor 42 may add, for example, a training for enhancing or maintaining the physical function of grasping an object.

### 3. EFFECTS AND LIKE

As described above, the determination method performed by a computer includes: instructing the target person to perform the specific action (S11 in FIG. 2); capturing an image that includes, as a subject, the target person performing the specific action (S12); estimating the skeletal model of the target person in the image based on the image captured (S13); setting a plurality of three-dimensional regions around the skeletal model based on the positions of a plurality of skeletal points in the skeletal model estimated (S14); identifying, among the plurality of three-dimensional regions set, a three-dimensional region where the skeletal point of a wrist of the target person is located in the specific action (S15); and determining the state of activities of daily living of the target person based on the three-dimensional region identified (S16).

In the determination method as described above, among the three-dimensional regions set around the skeletal model, the three-dimensional region where the skeletal point of the wrist of the target person is located in the specific action is identified, and thus it is possible to easily and accurately determine the state of activities of daily living of the target person.

For example, in the determination method, in the determining (S16), the state of activities of daily living of the target person is determined by determining, based on database 45 in which the specific action, the three-dimensional region where the wrist is located in the specific action, and the activity of daily living corresponding to the specific action are stored in association with each other, whether the three-dimensional region identified in the identifying (S15) matches the three-dimensional region stored in database 45.

In the determination method as described above, whether the three-dimensional region where the skeletal point of the wrist of the target person performing the specific action is located matches the three-dimensional region stored in database 45 in association with the specific action is identified, and thus it is possible to easily and accurately determine the state of activities of daily living of the target person.

For example, in the determination method, the capturing (S12) includes capturing moving images that include a plurality of images each being the image, and in the estimating (S13), the skeletal model in each of the plurality of images included in the moving images is estimated based on the moving images.

In the determination method as described above, based on the skeletal model in the moving images including the target person performing the specific action as the subject, the skeletal model corresponding to the movement of the target person performing the specific action can be estimated, and thus it is possible to set a plurality of three-dimensional regions according to the movement of the target person.

For example, in the determination method, the estimating includes estimating a two-dimensional skeletal model of the target person based on the image, and estimating a three-dimensional skeletal model of the target person based on the two-dimensional skeletal model estimated using a learned model that is a learned machine learning model, and in the setting, the plurality of three-dimensional regions are set based on the three-dimensional skeletal model.

In the determination method as described above, the three-dimensional skeletal model can be estimated using the learned model with the two-dimensional skeletal model in the image used as an input, and thus it is possible to determine the state of activities of daily living of the target person based on the image (or moving images) obtained from one camera 20.

For example, in the determination method, in the setting (S14), the plurality of three-dimensional regions are set around the skeletal model with one of the plurality of skeletal points in the skeletal model used as a base point, in a side view of the target person, the plurality of three-dimensional regions are included in any one of back surface region A3 on a back surface side of the target person, front surface region A2 on a front surface side of the target person, or forward region A1, back surface region A3 and front surface region A2 are provided adjacent to each other through first reference axis Z1 in a longitudinal direction that extends from the head of the target person to the legs of the target person and passes through the base point, forward region A1 is provided on a forward side of the target person to be adjacent to the front surface region, in a front view of the target person, each of back surface region A3, front surface region A2, and forward region A1 includes left side region B2 and right side region B1 of the target person that are provided adjacent to each other through second reference axis Z2 in the longitudinal direction which passes through the base point, and each of left side region B2 and right side region B1 includes a predetermined number of regions divided in a lateral direction orthogonal to the longitudinal direction from the head of the target person to the legs of the target person.

In the determination method as described above, the size and the position of the three-dimensional region where the wrist of the target person is located in the specific action are set according to activities of daily living of the target person, and thus it is possible to more accurately determine the state of activities of daily living of the target person.

For example, in the determination method, in first reference axis Z1, the skeletal point of the neck of the target person and the skeletal point of the waist of the target person each are used as the base point, in second reference axis Z2, the skeletal point of the neck of the target person and the skeletal point of an elbow of the target person each are used as the base point, and the setting (S14) includes setting, in the side view of the target person, first distance L1 from the skeletal point of the elbow of the target person to a tip of a hand of the target person as width W1 of each of back surface region A3, front surface region A2, and forward region A1, and setting, in the front view of the target person, a distance twice second distance L2 from the skeletal point of the neck of the target person to the skeletal point of a shoulder of the target person as width W2 of each of left side region B2 and right side region B1.

In the determination method as described above, the widths (a width and a depth in the front view) of a plurality of three-dimensional regions are set based on the positions of skeletal points, and thus, for example, even when the height of the target person is the same, a plurality of three-dimensional regions can be set according to the skeleton.

For example, the determination method further includes: presenting (S21 in FIG. 3), to the user, the state of activities of daily living of the target person determined in the determining; and receiving (S22) an instruction about an operation to be performed by the user, the determining (S16 in FIG. 2) includes extracting (S23) information about the state of activities of daily living of the target person based on the instruction of the user received in the receiving (S22), and the presenting (S21) includes presenting (S24) the information extracted in the determining (S23) to the user. For example, in the determination method, the information about the state of activities of daily living includes at least one of the three-dimensional skeletal model of the target person performing the specific action, the result of the determination of the physical function of the target person, or the detail of training to be proposed to the target person.

In the determination method as described above, it is possible to extract, based on the instruction of the user, information necessary for the user from the information about the state of activities of daily living of the target person to present the information to the user.

For example, in the determination method, the information about the state of activities of daily living includes the physical function of the target person, and the physical function of the target person is determined based on the state of at least one of an action of opening and closing a hand of the target person or an action of opposing fingers of the target person.

In the determination method as described above, whether an action accompanied by the movement of fingers of the target person can be performed is determined, and thus it is possible to determine, for example, whether the target person can grasp an object, with the result that it is possible to more accurately determine the state of activities of daily living of the target person.

A determination device includes: instructor 36 that instructs a target person to perform a specific action; camera 20 that captures an image which includes, as a subject, the target person performing the specific action; estimator 42b that estimates a skeletal model of the target person in the image based on the image captured; setter 42c that sets a plurality of three-dimensional regions around the skeletal model based on the positions of a plurality of skeletal points in the skeletal model estimated; identifier 42d that identifies, among the plurality of three-dimensional regions set, a three-dimensional region which includes the skeletal point of a wrist of the target person in the specific action; and determiner 42e that determines the state of activities of daily living of the target person based on the three-dimensional region identified.

In the determination device as described above, among the three-dimensional regions set around the skeletal model, the three-dimensional region where the skeletal point of the wrist of the target person is located in the specific action is identified, and thus it is possible to easily and accurately determine the state of activities of daily living of the target person.

Determination system 10 includes: information terminal 30; and server device 30 that is connected to information terminal 30 via communication, information terminal 30 includes: second communicator 31b that communicates with server device 40; instructor 36 that instructs the target person to perform the specific action; and camera 20 that captures the image which includes, as the subject, the target person performing the specific action, and server device 40 includes: estimator 42b that estimates the skeletal model of the target person in the image based on the image captured by camera 20; setter 42c that sets a plurality of three-dimensional regions around the skeletal model based on the positions of a plurality of skeletal points in the skeletal model estimated; identifier 42d that identifies, among the plurality of three-dimensional regions set, a three-dimensional region which includes the skeletal point of a wrist of the target person in the specific action; and determiner 42e that determines the state of activities of daily living of the target person based on the three-dimensional region identified.

In determination system 10 as described above, among the three-dimensional regions set around the skeletal model, the three-dimensional region where the skeletal point of the wrist of the target person is located in the specific action is identified, and thus it is possible to easily and accurately determine the state of activities of daily living of the target person.

### OTHER EMBODIMENTS

Although the embodiment has been described above, the present invention is not limited to the embodiment described above.

In the embodiment described above, processing performed by a specific processor may be performed by another processor. The order of a plurality of processing steps may be changed, and a plurality of processing steps may be performed in parallel with each other.

In the embodiment described above, constituent elements may be realized by executing software programs suitable for the constituent elements. A program executor such as a CPU or a processor may read and execute software programs recorded in a recording medium such as a hard disk or a semiconductor memory to realize the constituent elements.

The constituent elements may be realized by hardware. The constituent elements may be circuits (or integrated circuits). These circuits may form one circuit as a whole or may be separate circuits. These circuits may be general-purpose circuits or dedicated circuits.

The overall or specific form of the present invention may be realized by a system, a device, a method, an integrated circuit, a computer program, or a computer-readable recording medium such as a CD-ROM. The overall or specific form of the present invention may be realized by any combination of a system, a device, a method, an integrated circuit, a computer program, and a recording medium.

For example, the present invention may be realized as a determination method, may be realized as a program for causing a computer to perform a determination method, or may be realized as a non-transitory computer-readable recording medium in which the program as described above is recorded.

Although in the embodiment described above, the example is shown where the determination system includes the camera, the information terminal, and the server device, the determination system may be realized as a single device such as an information terminal or may be realized by a plurality of devices. For example, the determination system may be realized as a client server system. When the determination system is realized by a plurality of devices, there is no limitation as to how the constituent elements included in the determination system described in the above embodiment are allocated to the devices.

Embodiments obtained by performing various types of variations conceived by a person skilled in the art on the embodiments and embodiments realized by arbitrarily combining the constituent elements and the functions in the embodiments without departing from the spirit of the present invention are also included in the present invention.

### REFERENCE SIGNS LIST

1 target person
10 determination system
20 camera
30 information terminal
31b second communicator
34 receptor
35 presenter
36 instructor
40 server device
42b estimator
42c setter
42d identifier
42e determiner
43 storage
44 learned model
45 database
Z1 first reference axis
Z2 second reference axis
A1 forward region
A2 front surface region
A3 back surface region
B1 right side region
B2 left side region
L1 first distance
L2 second distance
W1 width
W2 width

1. A determination method performed by a computer, the determination method comprising:
instructing a target person to perform a specific action;
capturing an image that includes, as a subject, the target person performing the specific action;
estimating a skeletal model of the target person in the image based on the image captured;
setting a plurality of three-dimensional regions around the skeletal model based on positions of a plurality of skeletal points in the skeletal model estimated;
identifying, among the plurality of three-dimensional regions set, a three-dimensional region where a skeletal point of a wrist of the target person is located in the specific action; and
determining a state of activities of daily living of the target person based on the three-dimensional region identified.

2. The determination method according to claim 1,
wherein in the determining, the state of activities of daily living of the target person is determined by determining, based on a database in which the specific action, a three-dimensional region where the wrist is located in the specific action, and an activity of daily living corresponding to the specific action are stored in association with each other, whether the three-dimensional region identified in the identifying matches the three-dimensional region stored in the database.

3. The determination method according to claim 1,
wherein the capturing includes capturing moving images that include a plurality of images each being the image, and
in the estimating, the skeletal model in each of the plurality of images included in the moving images is estimated based on the moving images.

4. The determination method according to claim 1,
wherein the estimating includes:
estimating a two-dimensional skeletal model of the target person based on the image; and
estimating, based on the two-dimensional skeletal model estimated, a three-dimensional skeletal model of the target person using a learned model that is a learned machine learning model, and
in the setting, the plurality of three-dimensional regions are set based on the three-dimensional skeletal model.

5. The determination method according to claim 1,
wherein in the setting, the plurality of three-dimensional regions are set around the skeletal model with one of the plurality of skeletal points in the skeletal model used as a base point,
in a side view of the target person, the plurality of three-dimensional regions are included in any one of a back surface region on a back surface side of the target person, a front surface region on a front surface side of the target person, or a forward region, the back surface region and the front surface region being provided adjacent to each other through a first reference axis in a longitudinal direction that extends from a head of the target person to legs of the target person and passes through the base point, the forward region being provided on a forward side of the target person to be adjacent to the front surface region,
in a front view of the target person, each of the back surface region, the front surface region, and the forward region includes a left side region and a right side region of the target person that are provided adjacent to each other through a second reference axis in the longitudinal direction which passes through the base point, and
each of the left side region and the right side region includes a predetermined number of regions divided in a lateral direction orthogonal to the longitudinal direction from the head of the target person to the legs of the target person.

6. The determination method according to claim 5,
wherein the first reference axis is a normal to ground that passes through a skeletal point of a neck of the target person in the side view of the target person,
the second reference axis is a normal to the ground that passes through the skeletal point of the neck of the target person in the front view of the target person, and
the setting includes:
setting, in the side view of the target person, a first distance from the skeletal point of the elbow of the target person to a tip of a hand of the target person as a width of each of the back surface region, the front surface region, and the forward region; and
setting, in the front view of the target person, a distance twice a second distance from the skeletal point of the neck of the target person to a skeletal point of a shoulder of the target person as a width of each of the left side region and the right side region.

7. The determination method according to claim 1, further comprising:
presenting, to a user, the state of activities of daily living of the target person determined in the determining; and
receiving an instruction about an operation to be performed by the user,
wherein the determining includes extracting information about the state of activities of daily living of the target person based on the instruction of the user received in the receiving, and
the presenting includes presenting the information extracted in the determining to the user.

8. The determination method according to claim 7,
wherein the information about the state of activities of daily living includes at least one of a three-dimensional skeletal model of the target person performing the specific action, a result of a determination of a physical function of the target person, or a detail of training to be proposed to the target person.

9. The determination method according to claim 8,
wherein the information about the state of activities of daily living includes the physical function of the target person, and
the physical function of the target person is determined based on a state of at least one of an action of opening and closing a hand of the target person or an action of opposing fingers of the target person.

10. A determination device comprising:
an instructor that instructs a target person to perform a specific action;
a camera that captures an image which includes, as a subject, the target person performing the specific action;
an estimator that estimates a skeletal model of the target person in the image based on the image captured;
a setter that sets a plurality of three-dimensional regions around the skeletal model based on positions of a plurality of skeletal points in the skeletal model estimated;
an identifier that identifies, among the plurality of three-dimensional regions set, a three-dimensional region which includes a skeletal point of a wrist of the target person in the specific action; and
a determiner that determines a state of activities of daily living of the target person based on the three-dimensional region identified.

11. A determination system comprising:
an information terminal; and
a server device that is connected to the information terminal via communication,
wherein the information terminal includes:
a communicator that communicates with the server device;
an instructor that instructs a target person to perform a specific action; and
a camera that captures an image which includes, as a subject, the target person performing the specific action, and
the server device includes:
an estimator that estimates a skeletal model of the target person in the image based on the image captured by the camera;
a setter that sets a plurality of three-dimensional regions around the skeletal model based on positions of a plurality of skeletal points in the skeletal model estimated;
an identifier that identifies, among the plurality of three-dimensional regions set, a three-dimensional region which includes a skeletal point of a wrist of the target person in the specific action; and
a determiner that determines a state of activities of daily living of the target person based on the three-dimensional region identified.
